(19) United States
(12) Patent Application Publication
(10) Pub. No.: US 2015/0110758 A1
(43) Pub. Date: Apr. 23, 2015

(54) TREATMENT OF NEUROCUTANEOUS SYNDROME, INCLUDING COMPOSITIONS, METHODS AND USES THEREOF

(71) Applicant: Omar M. Amin, Scottsdale, AZ (US)

(72) Inventor: Omar M. Amin, Scottsdale, AZ (US)

(21) Appl. No.: 14/589,350

(22) Filed: Jan. 5, 2015

Related U.S. Application Data
(63) Continuation-in-part of application No. 13/367,629, filed on Feb. 7, 2012.
Application No. 13/367,629 is a continuation-in-part of application No. 13/623,558, filed on Sep. 20, 2012.

(51) Int. Cl. A61K 31/352; A61K 36/38; A61K 45/06

(52) U.S. Cl. CPC A61K 31/352; A61K 45/06; A61K 36/38
USPC 424/93.45; 424/725; 424/94.1; 424/769; 424/745; 424/778; 424/771; 424/776; 424/758; 424/773; 424/740; 424/744; 424/736; 424/738; 424/195.17; 424/750; 424/739; 424/756; 424/757; 424/760; 424/764; 424/746; 424/641; 424/682; 424/692; 424/93.4; 424/702; 514/690; 514/47; 424/747; 424/727

(57) ABSTRACT

A treatment protocol for use in the treatment of neurocutaneous syndrome is described and comprises at least one xanthone component, which may comprise at least one mangosteen component, and at least one detoxification component. Additional embodiments comprise at least one herbal component, at least one vitamin component, or a combination thereof. A method of treating a patient having neurocutaneous syndrome is described herein and comprises: providing at least one xanthone component, which may comprise at least one mangosteen component, providing at least one xanthone component, which may comprise at least one mangosteen component, to the patient, and administering the at least one detoxification component to the patient. Additional methods steps may include providing and administering at least one herbal component, providing and administering at least one vitamin component or a combination thereof.

This United States Utility Application is a Continuation In Part Application that claims priority to U.S. Utility application Ser. No.: 13/367629 and U.S. Utility application Ser. No.: 13/623558, both of which are commonly owned and incorporated herein by reference in their entirety.

### FIELD OF THE SUBJECT MATTER

The field of the subject matter is treatment of neurocutaneous syndrome, which is also known as Morgellons, including the compositions, methods and uses thereof.

### BACKGROUND

The terms “Morgellons” and “Neurocutaneous Syndrome” or NCS, as characterized by Dr. Omar Amin (2001-2009), are used interchangeably, yet cautiously, as their symptoms are very similar. While the etiological agent(s) and remedies of Morgellons have never been identified, these factors for NCS have been well-researched and published in refereed medical journals (see References Cited Section herein), and patients have been successfully helped.

Researchers in this area have recognized that patients experience dermatological abnormalities (elevated itchy skin sores that may develop into mucoid lesions) and neurological symptoms (movement, pin prick or crawling sensations) caused by toxic exposures to a wide variety of environmental factors. Those factors include, but are not limited to, incompatible dental materials, toxic fumes in the work place, insecticides or allergenic sprays, household chemicals, implants, recreational drugs, e.g., crystal methamphetamine and/or cocaine, medications, creams, hot sulfur/mineral springs, and any other environmental exposures to which the patient is allergic.

To date, no one has been able to develop and utilize a treatment protocol, including components and supplements directed specifically to treating NCS and the symptoms related to NCS. To this end, it would be desirable to develop, produce and utilize a formulation, formulations and related protocols that can easily and effectively treat NCS and the related symptoms, while at the same time minimizing pain and discomfort of the patient.

### SUMMARY OF THE SUBJECT MATTER

A treatment protocol for use in the treatment of neurocutaneous syndrome is described and comprises at least one xanthone component, which may include at least one mangosteen component, and at least one detoxification component. Additional embodiments comprise at least one herbal component, at least one vitamin component, or a combination thereof.

A method of treating a patient having neurocutaneous syndrome is described herein and comprises: providing at least one xanthone component, which may include at least one mangosteen component, providing at least one detoxification component, administering the at least one xanthone component to the patient, and administering the at least one detoxification component to the patient. Additional methods steps may include providing and administering at least one herbal component, providing and administering at least one vitamin component or a combination thereof.

### BRIEF DESCRIPTION OF THE FIGURES

FIG. 1 shows a contemplated method for the treatment of neurocutaneous syndrome, comprising a method 100 of treating a patient having neurocutaneous syndrome comprises: providing at least one xanthone component 110, which may include at least one mangosteen component, providing at least one detoxification component 120, administering the at least one xanthone component to the patient 130, and administering the at least one detoxification component to the patient 140. Additional methods steps may include providing and administering at least one herbal component, providing and administering at least one vitamin component or a combination thereof 150.

FIG. 2 shows a contemplated method for the treatment of neurocutaneous syndrome, comprising a method 200 of treating a patient having neurocutaneous syndrome comprises: providing at least one mangosteen component 210, providing at least one detoxification component 220, administering the at least one mangosteen component to the patient 230, and administering the at least one detoxification component to the patient 240. Additional methods steps may include administering a targeted questionnaire 250, administering a blood bio-compatibility test 260, administering massage therapy to the patient 270, which may include lymph drainage massage therapy, providing and administering at least one herbal component, providing and administering at least one vitamin component or a combination thereof (shown in FIG. 1), or a combination thereof.

### DETAILED DESCRIPTION

As described herein, a successful treatment protocol, including components and supplements directed specifically to treating NCS and the symptoms related to NCS has been developed. The Examples section will provide a detailed example of a contemplated treatment protocol and additional component parts. As also will be described herein, formulation, formulations and related protocols and methods that can easily and effectively treat NCS and the related symptoms, while at the same time minimizing pain and discomfort of the patient, have been developed and are being successfully utilized in practice.

Specifically, a treatment protocol for use in the treatment of neurocutaneous syndrome comprises at least one xanthone component, which may include at least one mangosteen component, and at least one detoxification component. Additional embodiments comprise at least one herbal component, at least one vitamin component, or a combination thereof.

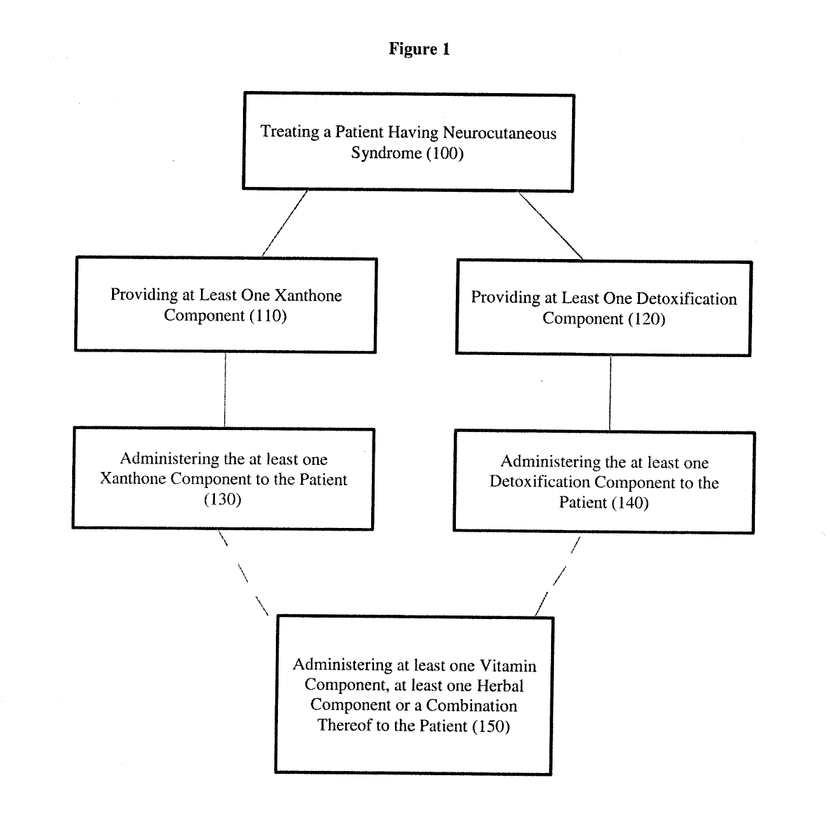

In addition, and as shown in FIG. 1, a method 100 of treating a patient having neurocutaneous syndrome comprises: providing at least one xanthone component 110, which may include at least one mangosteen component, providing at least one detoxification component 120, administering the at least one xanthone component to the patient 130, and administering the at least one detoxification component to the patient 140. Additional methods steps may include providing and administering at least one herbal component, providing and administering at least one vitamin component or a combination thereof 150.

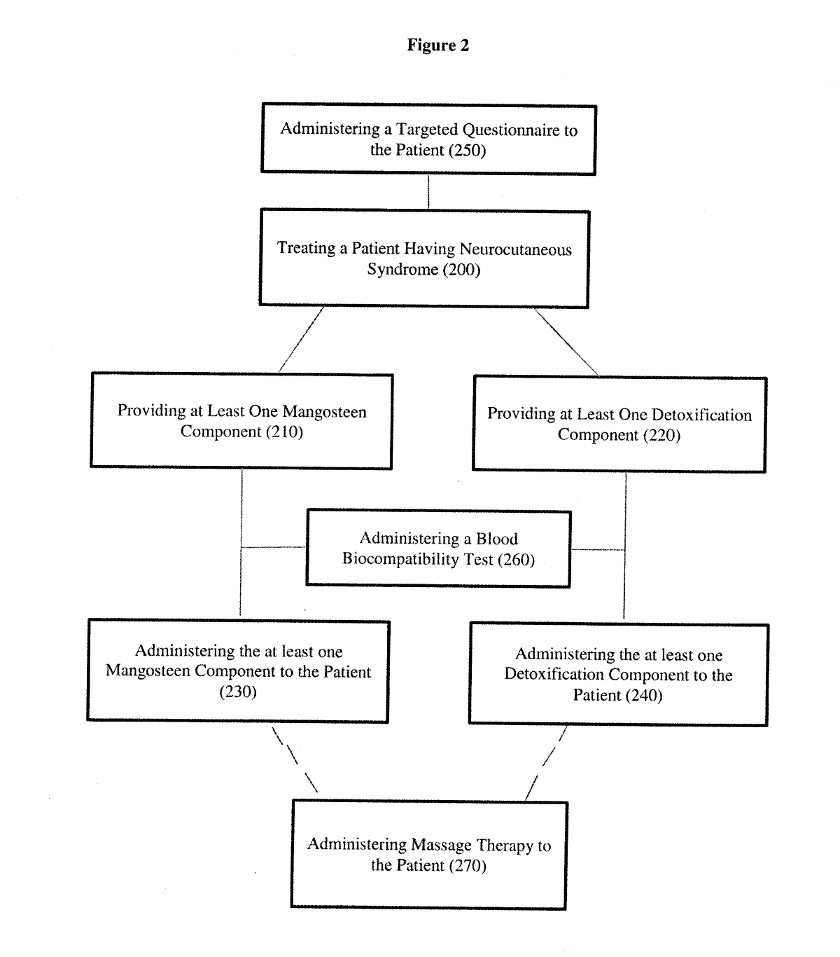

As shown in FIG. 2, a method 200 of treating a patient having neurocutaneous syndrome comprises: providing at least one mangosteen component 210, providing at least one detoxification component 220, administering the at least one mangosteen component to the patient 230, and administering the at least one detoxification component to the patient 240. Additional methods steps may include administering a targeted questionnaire 250, administering a blood bio-compatibility test 260, administering massage therapy to the patient 270, which may include lymph drainage massage therapy, providing and administering at least one herbal component, providing and administering at least one vitamin component or a combination thereof (shown in FIG. 1), or a combination thereof.

It should be understood that “administering” in the context of this application, for example: administering the at least one xanthone component to the patient in a dosing regimen that is based on the combined set of results from the combination of the targeted questionnaire results and the biocompatibility test results means that something is being administered. In other words, the provision can be read: “administering” . . . the “component” in a “dosing regimen”—the “dosing regimen” being based on the results from the “combination of the targeted questionnaire results and the biocompatibility test results”. The phrases that use the term “administering” can also be read, for example in context, as follows: “administering the at least one xanthone component to the patient in a dosing regimen, wherein the dosing regimen is based on the combined set of results from the combination of the targeted questionnaire results and the biocompatibility test results”.

Treatment protocols and methods of treating patients, as disclosed herein, include at least one xanthone component, which may include at least one mangosteen component. The mangosteen fruit, particularly the rind, or pericarp, has been used in traditional folk medicine for centuries. It is well known in Asia, where it is often called “The Queen of Fruits”, but is unavailable in the United States. Folk medicine has long used the mangosteen peel for a variety of ailments, used differently in different parts of Asia. As disclosed herein, the “at least one mangosteen component” refers to the extract of the fruit, the extract of the rind, the fruit, the juice from the fruit, the rind, or a combination thereof.

Research scientists are now discovering that there is valid science behind the folk medicine traditions and the answer seems to be in the mangosteen's compounds called xanthones. There have been 43 xanthones discovered in the fruit thus far (mostly in the rind); some unique only to the mangosteen. Xanthones are potent anti-oxidants and are used for their anti-toxic benefits; they have been found to have potent anti-inflammatory, anti-bacterial, anti-viral, anti-tumor and anti-fungal properties; they help with neurological and cardiovascular disorders and have other healing properties as well. These properties have prompted health professionals to utilize the mangosteen in a variety of conditions, including pain, gastrointestinal problems, dermatologic diseases (including eczema, skin sores and lesions, etc.), musculoskeletal conditions, dental/oral disorders and much more. The mangosteen is one of nature's only readily available sources of xanthones known thus far. In contemplated embodiments, the xanthone component or the mangosteen component may be administered in any suitable amount, depending on the needs of the patient, in order to treat NCS. Amounts that are not suitable to treat NCS are not contemplated herein.

Additionally, mangosteen was used traditionally for its anti-parasitic effects: Malaria, amoebic dysentery and syphilis are caused by parasites. Asian healers have used mangosteen for centuries to deal with those illnesses. In South America, mangosteen is also used to cure intestinal worm infestations. Laboratory research to confirm these practices, however, is not yet available at this time.

As disclosed, contemplated treatment protocols and methods of treating patients include at least one detoxification component. In some embodiments, the detoxification component may either be or comprise a mangosteen component. In other embodiments, the at least one detoxification component may comprise additional components other than mangosteen or its derivatives.

Contemplated treatment protocols and methods of treating patients with parasitic infections may also include providing and utilizing at least one of the following herbal components in a suitable amounts: Oregano Leaf, Clove Flower, Black Walnut Husk, Peppermint Leaf, Black Cumin Seed, Winter Melon Seed, Gentian Root, Wormwood Bark, Hyssop Leaf, Cramp Bark, Thyme Leaf, Fennel Seed, Pumpkin Seed, Caprylic Acid, Pagoda Fruit, Rosemary, Aloe Vera, Betel Nut Palm, Papaya, Citrus Extract, Pomegranate, Rangoon Creeper, Tansy, Psyllium Seed Husk, Atlantic Kelp, Corn Silk, Fo-Ti, Chinese Rhubarb, Black Cumin Seed, Cinnamon Bark, Fennel Seed, Ginger Root, Orange Peel Extract, Clove Bulb, Cascara Sagrada, Slippery Elm Bark, Molasses, Carob, Rhubarb Root, Thyme Leaf, Clove Bulb, Cayenne Fruit, Rosemary Leaf, Alfalfa Leaf, Licorice Root, Chamomile, Grapefruit Seed, Echinacea, Chicory Root, Corn Silk, Safflower Oil Powder, Sage or a combination thereof. In some contemplated embodiments, a combination of at least some of these components is packaged in three anti-parasitic formulas that may be referred to as “Freedom/Cleanse/Restore”.

Contemplated detoxification formulas, formulations or combinations of components comprise at least one of the following: beta vulgaris, coenzyme A, coenzyme Q-10, berberis vulgaris or nux vomica. Contemplated detoxification formulas and products that can be utilized as at least part of the at least one herbal component includes a HEEL Detox kit, Ubichinon, Coenzyme A or Coenzyme Q-10, the Bio Cytotox Phase, the Bio Ubichinon Compositum, the BodyAnew Detox Package or a combination thereof. The Bio Cytotox Phase, the Bio Ubichinon Compositum, the BodyAnew Detox Package are manufactured by Desert Biologicals.

The HEEL Detox kit comprises three homeopathic preparations: berberis-homaccord (100 ml contain: medicinal ingredients: Berberis vulgaris D2, D10, D30, D200 0.4 g each; Colocynthis D2, D10, D30, D200; Veratrum album D3, D10, D30, D200 0.3 g each. Non-medicinal ingredients: ethanol, purified water), 100 ml contains: medicinal ingredients: Juglans regia D3 5g; Myosotis arvensis D3 5 g; Scrophularia nodosa D3 5 g; Teucrium scorodonia D3 5 g; Veronica officinalis D3 5 g; Geranium robertianum D4 10 g; Urtica urens D4 10 g; Equisetum hyemale D4 5 g; Fumaria officinalis D4 5 g; Natrium sulfuricum D4 5 g; Pinus sylvestris D4 5 g; Gentiana lutea D5 5 g; Apis mellifica D6 10 g; Fucus vesiculosus D6 10 g; Sarsaparilla D6 5 g; Calcium phosphoricum D12 5 g. Non-medicinal ingredients: ethanol, purified water); and nux vomica-homaccord (100 ml contains: medicinal ingredients: Bryonia D2, D6, D10, D15, D30, D200, D1000, Nux vomica D2, D10, D15, D30, D200, D1000 0.2 g each, Colocynthis D3, D10, D30, D200, Lycopodium clavatum D3, D10, D30, D200, D1000 0.3 g each. Non-medicinal ingredients: ethanol, purified water). It should be understood that other components and methods of detoxification can also be used.

The Bio Cytotox Phase stimulates defensive mechanisms against toxins to reactivate the blocked and defective enzymatic functions in degenerative diseases. Bio Cytotox Phase includes as ingredients: Colchicum autumnale 4×, Conium maculatum 4×, Hydrastis canadensis 4×, Podophyllum peltatum 5×, Vaccinium myrtillus 4×, Ascorbic acid 6×, Galium aparine 6×, Nicotinamidum 6×, Pyridoxinum hydrochloricum 6×, Riboflavinum 6×, Sarcolacticum acidum 6X, Thiaminum hydrochloricum 6×, alpha-Lipoicum acidum 833 , Hydroquinone 3C, Manganum phosphoricum 15×, Natrum oxalaceticum 8×, Sulphur 8×, Anthraquinone 10×, Acetylsalicylicum acidum 10×, ATP 1033 , Coenzyme A 10×, Co-enzyme Q-10 10×, Histaminum hydrochloricum 10×, Magnesium gluconicum 10×, Nadidum 10×, Naphthoquinone 10×, Norepinephrine 6×12×30×200×12C 30C 60C 200C, and Morgan Gaertner 30C.

The Bio Coenzyme Compositum stimulates blocked enzymatic systems in both degenerative diseases and defective enzymatic functions. It is a complex homeopathic remedy indicated for the relief of exhaustion, fatigue and feelings of low energy. Its ingredients include: Beta vulgaris 4×, alpha-Lipoicum acidum 8×, Ascorbic acid 6×, Cysteinum 6×, Magnesia oroticum 8×, Manganum phosphoricum 16×, Natrum oxalaceticum 6×, Nicotinamidum 6×, Pulsatilla (Vulgaris) 6×, Pyridoxinum hydrochloricum 6×, Riboflavinum 6×, Thiaminum hydrochloricum 6×, alpha-Ketoglutaricum acidum 8×, Cerium oxalicum 8×, cis-Aconiticum acidum 8×, Citricum acidum 8×, Coenzyme A 8×, Fumaricum acidum 8×, Malic acid 8×, Nadidum 8×, Natrum pyruvicum 8×, Succinicum acidum 8×, ATP 10×, Baryta oxaliuccinicum 30C, Hepar sulphuris calcareum 10×, Sulphur 10×, Adrenalinum 6×12×30×200×12C 30C 60C 200C, and Morgan Bacillus Pure 30C.

The Body Anew Detox package that was formerly known as the HEEL Detox kit is a homeopathic liquid remedy designed for drainage and detoxification from internal toxins affecting the internal organ systems. It is the first and most potent line of defense against toxic chemicals that restores normal balanced immune functions. Ingredients of the Body Anew Detox package include: Active Ingredients: Berberis Vulgaris 2×, 10×, 30×200× (0.2 ml), Colocynthis 3× (1.5 ml), 10×, 30×, 200× (0.15 ml each), Veratrum Album 3×, 10×, 30×, 200× (0.15 ml), Aranea Diadema 6× (2.5 ml), Calcarea Phosphorica 12× (2.5 ml), Equisetum Hyemale 4× (2.5 ml), Ferrum lodatum 12× (5 ml), Fumaria Officinalis 4× (2.5 ml), Gentiana Lutea 5× (2.5 ml), Geranium Robertianum 4× (5 ml), Levothyroxine 12× (2.5 ml), Myosotis Arvensis 3× (2.5 ml), Nasturtium Aquaticum 4× (5 ml), Natrum Sulphuricum 4× (2.5 ml), Pinus Sylvestris 4× (2.5 ml), Sarsaparilla 6× (2.5 ml), Scrophularia Nodosa 3× (2.5 ml), Teucrim Scorodonia 3× (2.5 ml), Veronica Officinalis 3× (2.5 ml), Bryonia Alba 3×, 6×, 10×, 15×, 30×, 200×, 1000× (0.1 ml Each), Colocynthis 3×, 10×, 30×, 200× (0.15 ml Each), Lycopodium Clavatum 3×, 10×, 30×, 200×, 1000× (0.15 ml Each), Nux Vomica 3×, 10×, 15×, 30×, 200×, 1000× (0.1 ml Each). Inactive Ingredients include: Ethyl Alcohol, and Purified Water USP.

Contemplated treatment protocols and methods of treating patients may also include providing and utilizing at least one vitamin component, which includes or may include one or more of the following components or ingredients in a suitable amount: vitamin B (all forms, including vitamin B12, B5 and B6), folic acid, minerals, such as zinc, calcium, magnesium oxide or citrate, vitamin E, vitamin C, Omega oils (including Omega 3 fish oil or lin seed oil), acidofilus, bifidus, lactobacillus, amino acids, digestive enzymes, selenium and L-glutathione. A contemplated daily dose of the at least one vitamin component may include:

Vitamin B12: 1-2 mg (methycobalamin)

Vitamin B6: 100 mg (pyridokal-5-phosphate)

Folic Acid: 200-300 mcg

Zinc: 30-50 mg

Vitamin E: 300-500 IU (dl-alpha tocopheryl acetate)

Vitamin B5: 500 mg (pantothenic acid)

Calcium: 200-400 mg (not carbonate)

Magnesium Oxide or Citrate: 300-500 mg

Time release Vitamin C: 1000-3000 mg

Omega 3 fish oil or lin seed oil

Acidofilus, Bifidus, Lactobacillus

Amino Acids

Digestive Enzymes

Selenium: 100-200 mcg

L Glutathione: 100-150 mg (50-250, suitable)

All patients that comply, at least partly and ideally fully, with contemplated embodiments and programs have recovered. Neurocutaneous Syndrome (NCS), a newly discovered toxicity syndrome is characterized by neurological and dermatological disorders as well as systemic and related dysfunctions.

As disclosed earlier, patients experience, among other symptoms, pin-prick movement sensations and in later stages, itchy cutaneous lesions that may invite various opportunistic infections (insects, worms, fungus, pathogenic bacteria, among others) often confused as causative agents of the syndrome.

The toxicity of dental chemicals, compounding factors and case histories were discussed and management protocols researched at the Parasitology Center, Inc. (PCI) were proposed. Components of calcium hydroxide sealants and liners (Dycal, Life, and Sealapex) and at least 644 other dental chemicals, have been identified as a source of the observed symptoms. NCS patients often confuse the movement sensations, itchy skin and related symptoms with parasitic infections and seek medical help under this assumption. Invariably, they are diagnosed with and treated for other etiologies often including arthropod infestation and/ or mental conditions such as psychosis and delusional parasitosis. Patients are genuine clinical cases who should not be further compromised by inaccurate diagnosis, wrongly medicated or subjected to psychological treatment in mental health care facilities.

### EXAMPLES

### Example 1

### A Contemplated Treatment Protocol

A contemplated treatment protocol has been developed and designed to identify, analyze, treat and follow-up with a patient who has neurocutaneous syndrome or NCS. It should be understood that many of the components and methods disclosed herein are incorporated in this Example, along with other desirable components and methods steps.

### A Protocol for NCS Rehabilitation

Interviewing the patient.

Examining patient and verifying symptoms, history, and the presence of toxic dental materials. Documenting dermatological symptoms with photos.

Examinating dental and related records.

Administering a targeted questionnaire to the patient.

Documenting symptoms, dates and history.

Swabbing patient's skin for culture of bacteriological and fungal infections.

Establishing a diagnosis and providing a diagnostic report to patient.

Referring patient to a holistic dentist in patient's area of residence.

Initiating blood dental-bio compatibility testing.

Identifing compromised teeth and begin treatment; no more than 3 teeth restored in one month.

Supplementing with the at least one vitamin component during treatment; symptoms will get worse before they get better.

Making recommendations for additional nutritional remedies.

Initiating detoxification of organ systems by utilizing the at least one herbal component.

Instituting lymph drainage massage therapy, and laser therapy if needed.

Following up with patient.

The first three steps of the above-referenced protocol are self-explanatory and should be understood by anyone who is trained in dentistry or is a trained physician. The next step includes administering a targeted questionnaire to the patient. A contemplated questionnaire is found below.

### NCS Questionnaire

Mark the intensity of the following symptoms on a scale of 1-3. 1; mild, 2; moderate, 3; severe.

Use approximate date for “first observed” and use N/A, occasionally, often, and always for frequency.

Based on the protocol outlined above, along with the responses to the Questionnaire outlined herein, a detailed analysis of the clinical history of a random sample of 50 NCS patients (9 males, 41 females) was reported.

Symptoms were classified into six categories, neurological (sensory imbalances), dermatological (including opportunistic skin infections), systemic, oral, allergic and general. The most common symptoms in each of these categories in the same order are pin prick and crawling sensations, skin lesions and sores, respiratory and bowel disturbances, gum disease, sensitivities to light, noise and mold, and fatigue and insomnia. Symptoms were relatively similar in both sexes. These results were tabulated and their biological foundation explained. The misdiagnosis of NCS cases by medical professionals is discussed. NCS symptoms in toothless patients or those with dentures, and those on recreational drugs are described. Initially, over 360 dental toxins are placed in four major categories and their mode of action explained. Incubation period varied between a few hours to 28 years. Our protocol for rehabilitation is included. All patients following and completing our rehabilitation program have invariably recovered.

Early reports on this syndrome included the description of a case with many facial opportunistic infections from Oklahoma and the first naming and evaluation of the syndrome from three more cases, with a special reference to fibers and springtails (Collembola). By 2003, a comprehensive evaluation of NCS and establish the link to dental toxins as the causative agents was able to be provided. Amin clarified the nature of action of dental liners (bases) in the causation of NCS neurological and dermatological symptoms and provided the history of three NCS patients who have recovered following rehabilitation thus establishing a cause-effect relationship. Various versions of this landmark publication were subsequently published.

The above contributions were researched and published, and patients were successfully helped long before we discovered a similar clinical entity called Morgellons. The only difference is that we have done the research, established a causal relationship with dental toxins, developed a protocol, and successfully helped patients.

Most people have had dental work. Many have various degrees of sensitivity to some dental materials to which their bodies manifest varied intensities of symptoms. This epidemic-in-disguise has been routinely misunderstood by medical professionals who often label patients as delusional because of their unfortunate description of their neurological symptoms (actually caused by nerve damage) as having been caused by parasite infections. Amin specifically addressed this issue while discussing the clinical history of 24 NCS patients.

Of these patients, seven who have followed and completed the contemplated and disclosed protocol have experienced full recovery.

Amin provided an annotated list of about 400 dental materials that have been involved in the causation of NCS symptoms in patients that we have studied. Toxic ingredients common to all listed chemicals were classed in four categories. These categories are found in many more dental chemicals that were not reported in Amin's preliminary list. An overview of NCS made special reference to organ system symptomology in 50 patients of both sexes and all age groups, misdiagnoses, storage organs, sealants, drug involvement, incubation period, and recovery, with the discussion of five relevant cases. The personal perspectives of patients who have recovered from NCS have been presented by themselves.

Thus, specific embodiments, protocols and methods of the treatment of neurocutaneous syndrome have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification and claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES CITED

Amin, O. M. 1996. Facial cutaneous dermatitis associated with arthropod presence Explore! for the professional 7:62-64, incorporated herein in its entirety by reference.

Amin, O. M. 2001. Neuro-cutaneous Syndrome (NCS): a new disorder. Explore! for the Professional 10:55-56, incorporated herein in its entirety by reference.

Amin, O. M. 2003. On the diagnosis and management of Neuro-cutaneous Syndrome (NCS), a toxicity disorder from dental sealants. Explore! for the Professional 12:21-25, incorporated herein in its entirety by reference.

Amin, O. M. 2004 b. On the course of Neuro-cutaneous Syndrome (NCS) and its pseudo-diagnosis by medical professionals. Explore! for the Professional 13:4-9, incorporated herein in its entirety by reference.

Amin, O. M. 2006 b. An Overview of Neuro-cutaneous Syndrome (NCS) with a special reference to symptomology. Explore! for the Professional 15:41-49, incorporated herein in its entirety by reference.

Amin, O. M. 2007. The face of Neuro-cutaneous Syndrome (NCS): new cases, recovery, and perspectives. Explore! for the Professional 16:54-64, incorporated herein in its entirety by reference.

Keleher, J. W. 2008. Hell and Back Again, and Account of Morgellons Disease and it's Cure From a Former Sufferer. Explore! for the Professional 17:14-16.

Amin, O. M. 2009 Recovering from Morgellons and Neuro-cutaneous Syndrome (NCS): Patients & Perspectives. Explore for the Professional. 18:1-8, incorporated herein in its entirety by reference.

Amin, O. M.2010 In Their Own Words or: Symptoms of Morgellons and Neuro-Cutaneous Syndrome (NCS) Upon First Examination at the Parasitology Center, Inc. (PCI) 19:1-8, incorporated herein in its entirety by reference.

Amin, O. M. 2012 A Comprehensive Study of Dental Materials and Their Toxic Ingredients Associated with Neuro-Cutaneous Syndrome (NCS) and Morgellons, with Notes on Research Background.Explore! 21.1:14-23, incorporated herein in its entirety by reference.

Moongkarndi, P. et al. Antiproliferation, Antioxidation and Induction of Apoptosis by Garcinia Mangostana (Mangosteen) on SKBR3 Human Breast Cancer Cell Line. J. Ethnopharmacol. 2004; 90 (1):161-6.

Nakatani, K. et al. Inhibition of Cyclooxygenase and Prostaglandin E2 Synthesis by Gamma-Mangostin, a Xanthone Derivative in Mangosteen, in C6 Rat Glioma Cells. Biochem Pharmacol. 2002 Jan 1; 63(1):73-9.

Sakagami, Y. et al. Antibacterial Activity of Alpha-Mangostin against Vancomycin Resistant Enterococci (VRE) and Synergism with Antibiotics. Phytomedicine. 2005; 12(3):203-8.

Chen, S X. Active Constituents against HIV-1 Protease from Garcinia Mangostana. Planta Med 1996; 62 (4) 381-2.

Nabandit, V. et al. Inhibitory Effects of Crude Alpha Mangostin, a Xanthone Derivative, on Two Different Categories of Colon Preneoplastic Lesions Induced by 1, 2-dimethylhydrazine in the Rat. Asian Pac J. Cancer Prey. 2004; 5(4):433-8.

Dharmaratne, H R et al. A Geranylated Biphenyl Derivative from Garcinia Mangostana. Nat. Prod. Res. 2005 Apr; 19(3):239-43.

Nakatani, K. et al. Gamma Mangostin Inhibits Kinase Activity and Decreases Lipopolysaccharide-Induced Cyclooxygenase-2 Gene Expression in C6 Rat Glioma Cells. Molecular Pharmacol. Sep 2004; 66(3)667-674.

Jiang, D J et al. Pharmacological Effects of Xanthones as Cardiovascular Protective Agents. Cardiovascular Drug Rev. 2004; 22 (2):91-102.

Nakatani, K. et al. Inhibitions of Histamine Release and Prostaglandin E2 Synthesis by Mangosteen, a Thai Medicinal Plant. Biol. Pharm Bull. 2002; 25 (9):1137-41.

Begum, N, et al. Anti-ulcer and Antimicrobial Activities of Gartanin, a Xanthone from Garcinia Mangostana Linn. Bulletin Islam. 1982; 2 (20) 518-521.

Templeman, J. Frederick, Mangosteen, The X-Factor. Volume 3, pp. 37-38.

Templeman, J. Frederick, Mangosteen, The X-Factor. Volume 3, pp. 14-18.

Templeman, J. Frederick, Mangosteen, The X-Factor. Volume 3, Preface.

I claim:

1. A method of treating a patient having neurocutaneous syndrome caused by dental toxins, comprising:
providing at least one targeted questionnaire to the patient,
providing a blood dental biocompatibility test,
providing at least one xanthone component,
providing a detoxification component, wherein the detoxification component comprises at least one of the following: beta vulgaris, coenzyme A, coenzyme Q-10, berberis vulgaris or nux vomica;
administering the at least one targeted questionnaire to the patient to provide targeted questionnaire results,
administering a blood dental biocompatibility test to the patient to provide biocompatibility test results, wherein the targeted questionnaire results and the biocompatibility test results are combined to provide a set of results,
administering the at least one xanthone component to the patient in a dosing regimen that is based on the combined set of results from the combination of the targeted questionnaire results and the biocompatibility test results, and
administering the detoxification component to the patient in a dosing regimen that is based on the combined set of results from the combination of the targeted questionnaire results and the biocompatibility test results.

2. The method of claim 1, further comprising:
providing and administering at least one herbal component, providing and administering at least one vitamin component or a combination thereof.

3. The method of claim 1, wherein the at least one xanthone component comprises at least one mangosteen component.

4. The treatment protocol of claim 3, wherein the at least one mangosteen component comprises an extract of a mangosteen fruit.

5. The treatment protocol of claim 3, wherein the at least one mangosteen component comprises a suitable amount of the extract of the mangosteen fruit.

6. The treatment protocol of claim 3, wherein the at least one mangosteen component comprises mangosteen juice.

7. A method of treating a patient having neurocutaneous syndrome caused by dental toxins, comprising:
providing at least one targeted questionnaire to the patient,
providing a blood dental biocompatibility test,
providing at least one mangosteen component,
providing a detoxification component, wherein the detoxification component comprises at least one of the following: beta vulgaris, coenzyme A, coenzyme Q-10, berberis vulgaris or nux vomica;
administering the at least one targeted questionnaire to the patient to provide targeted questionnaire results,
administering a blood dental biocompatibility test to the patient to provide biocompatibility test results, wherein the targeted questionnaire results and the biocompatibility test results are combined to provide a set of results,
administering the at least one mangosteen component to the patient in a dosing regimen that is based on the combined set of results from the combination of the targeted questionnaire results and the biocompatibility test results, and
administering the at least one detoxification component to the patient in a dosing regimen that is based on the combined set of results from the combination of the targeted questionnaire results and the biocompatibility test results.

8. The method of claim 7, wherein the at least one mangosteen component comprises an extract of a mangosteen fruit.

9. The method of claim 7, wherein the at least one mangosteen component comprises a suitable amount of the extract of the mangosteen fruit.

10. The method of claim 7, wherein the at least one mangosteen component comprises mangosteen juice.

11. The method of claim 7, further comprising providing and administering massage therapy to the patient.

12. The method of claim 11, wherein massage therapy comprises lymph drainage massage therapy.

13. A treatment protocol for use in the treatment of neurocutaneous syndrome comprising:
administering to a patient in need thereof, at least one xanthone component;
and administering to a patient in need thereof, a detoxification component, wherein the detoxification component comprises at least one of the following: beta vulgaris, coenzyme A, coenzyme 0-10, berberis vulgaris or nux vomica.

14. The treatment protocol of claim 13, further comprising administering to a patient in need thereof, at least one herbal component, at least one vitamin component, or a combination thereof.

15. The treatment protocol of claim 13, wherein the at least one xanthone component comprises at least one mangosteen component.

16. The treatment protocol of claim 15, wherein the at least one mangosteen component comprises an extract of a mangosteen fruit.

17. The treatment protocol of claim 15, wherein the at least one mangosteen component comprises a suitable amount of the extract of the mangosteen fruit.

18. The treatment protocol of claim 15, wherein the at least one mangosteen component comprises mangosteen juice.

19. The treatment protocol of claim 14, wherein the at least one herbal component comprises Oregano Leaf, Clove Flower, Black Walnut Husk, Peppermint Leaf, Black Cumin Seed, Winter Melon Seed, Gentian Root, Wormwood Bark, Hyssop Leaf, Cramp Bark, Thyme Leaf, Fennel Seed, Pumpkin Seed, Caprylic Acid, Pagoda Fruit, Rosemary, Aloe Vera, Betel Nut Palm, Papaya, Citrus Extract, Pomegranate, Rangoon Creeper, Tansy, Psyllium Seed Husk, Atlantic Kelp, Corn Silk, Fo-Ti, Chinese Rhubarb, Black Cumin Seed, Cinnamon Bark, Fennel Seed, Ginger Root, Orange Peel Extract, Clove Bulb, Cascara Sagrada, Slippery Elm Bark, Molasses, Carob, Rhubarb Root, Thyme Leaf, Clove Bulb, Cayenne Fruit, Rosemary Leaf, Alfalfa Leaf, Licorice Root, Chamomile, Grapefruit Seed, Echinacea, Chicory Root, Corn Silk, Safflower Oil Powder, Sage or a combination thereof.

20. The treatment protocol of claim 14, wherein the at least one vitamin component comprises vitamin B, folic acid, minerals, such as zinc, calcium, magnesium oxide or citrate, vitamin E, vitamin C, Omega oils, acidofilus, bifidus, lactobacillus, amino acids, digestive enzymes, selenium, L-glutathione or a combination thereof.
